(19) United States
(12) Patent Application Publication
(10) Pub. No.: US 2020/0239629 A1
(43) Pub. Date: Jul. 30, 2020

(54) HINDERED POLYMERIC AMINES

(71) Applicant: 3V Sigma S.P.A., Milano (IT)

(72) Inventors: Ferruccio Berte', Bergamo (IT); Francesco Maestri, Bergamo (IT); Simone Del Sordo, Bergamo (IT)

(73) Assignee: 3V Sigma S.P.A., Milano (IT)

(21) Appl. No.: 16/845,226

(22) Filed: Apr. 10, 2020

Related U.S. Application Data
(63) Continuation of application No. 16/019,667, filed on Jun. 27, 2018, now abandoned.

(30) Foreign Application Priority Data
Jun. 30, 2017 (IT) 102017000073726

(51) Int. Cl. C08G 63/685; C08L 23/12; C07D 401/14; C08G 73/02; C08L 77/06; C08K 5/3492

(52) U.S. Cl. CPC C08G 63/6856; C08L 23/12; C08K 5/34922; C08G 73/0273; C08L 77/06; C07D 401/14

(57) ABSTRACT

Disclosed are polymer compounds wherein the main polymer chain is a polyester and the pendants are polyalkylpiperidine. The compounds of the invention, either alone or mixed with other known agents, are useful as stabilisers of polymers, especially polyolefins.

This Non-Provisional Application is a continuation application of U.S. application Ser. No. 16/019,667 filed Jun. 27, 2018 which claims priority to and the benefit from Italian Patent Application No. 102017000073726 filed on Jun. 30, 2017, the contents of which are incorporated herein by reference in their entirety.

The present invention relates to polyalkylpiperidine polymer compounds which impart high stability to various kinds of polymer materials, especially polyolefins, towards oxidative action and photodegradation.

### PRIOR ART

Polymers are known to be subject to deterioration due to the action of heat, light and oxygen; these factors cause loss of their mechanical properties, discolouring and other adverse effects.

In order to stabilise polymer materials, mainly towards the UV radiation in sunlight, various classes of compounds have been proposed, such as benzophenone and benzotriazole derivatives. The stability which these compounds give to polymers is acceptable, but insufficient to meet current practical needs, especially in the case of fibres, films and raffia based on olefin polymers.

The polyalkylpiperidine derivatives commonly called HALS (hindered amine light stabilisers) are much more effective, and there are numerous patents relating to them.

Examples of HALS are described in U.S. Pat. No. 4,530,950, DE 1,929,928, U.S. Pat. Nos. 3,640,928, 4,477,615, 4,233,412, 4,331,586, DE 2,636,144, DE 2,456,864, U.S. Pat. Nos. 4,315,859, 4,104,248, 4,086,204, 4,038,280, 4,476,302, 4,981,964 and EP 2,632,914,

Synergistic mixtures of HALS are described in U.S. Pat. Nos. 4,692,486, 4,863,981, 5,021,485, EP 0709426, EP 0728806 and WO 2012/153260.

However, there is still a need for further compounds or mixtures that possess high stabilising efficacy towards polymer materials.

Particularly useful polyalkylpiperidine compounds are those wherein the active units based on hindered amines are present as pendants of polymer chains.

### DESCRIPTION OF THE INVENTION

The present invention relates to novel polymer compounds wherein the main polymer chain is a polyester and the pendants are polyalkylpiperidino groups.

Polymer compounds wherein the main chain is a polyester, and the polyalkylpiperidine units are part of the chain, are already known and widely used.

Stabilisers of this type, and their use as light stabilisers in polymers, are described, for example, in U.S. Pat. No 4,233,412.

Trade names for this type of product, the CAS RN of which is 65447-77-0, are UVASORB HA22 (3V Sigma SpA), Tinuvin 622 (Basf), Lowilite 62 (Addivant) and Light Stabilizer 622, UV-622.

However, in these compounds the activity of the polyalkylpiperidine groups can be partly reduced by the fact that the groups active for light stabilisation are blocked in the polymer chain.

It is therefore desirable to manufacture and use polyester stabilising compounds wherein the groups active for radiation stabilisation are bonded to the main chain as pendants, and therefore more mobile and able to perform a better stabilising action.

A first aspect of the invention relates to compounds of general formula (I):

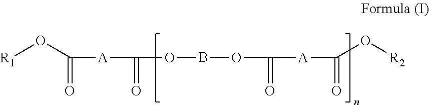

wherein:

n is an integer ranging from 2 to 100;

R₁ and R₂ are hydrogen or saturated, unsaturated, straight or branched C₁-C₁₈ alkyls or aromatic groups.

A is a C₁-C₂₀ alkylene or C₃-C₁₀ cycloalkylene group optionally containing one or more unsaturations or an aromatic group;

wherein “aromatic group” preferably means a phenylene or naphthalene ring, and B is a group of Formula (II):

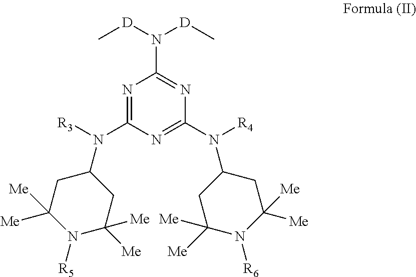

wherein:

D is a C₁-C₆ alkylene or isoalkylene group, optionally unsaturated;

R₃ and R_{4,} which may be the same or different, are hydrogen or C₁-C₈ alkyl;

R₅ and R_{6,} which may be the same or different, are hydrogen or straight or branched C₁-C₄ alkyl or an OG group wherein

G is hydrogen or saturated, unsaturated, straight or branched C₁-C₁₀ alkyl.

The preferred compounds of formula I are those wherein:

n is between 3 and 20,

A is a straight-chain C₁-C₂₀ alkylene group, preferably a —(CH₂)₈— group,

D is a —CH₂—CH₂—, —CH₂—CH₂—CH₂—, —CH(CH₃)—CH₂— or —CH₂—CH(CH₃)— group, preferably a —CH₂—CH₂—, —CH(CH₃)—CH₂— or —CH₂—CH(CH₃)— group

R₁ and R₂ are methyl or ethyl, and are preferably both methyl,

R₃ and R₄ are hydrogen or n-butyl, and are preferably both n-butyl,

R₅ and R₆ are hydrogen or methyl.

The polypiperidine polymer compounds of formula (I) can be obtained by a polycondensation reaction between an intermediate of general formula (III)

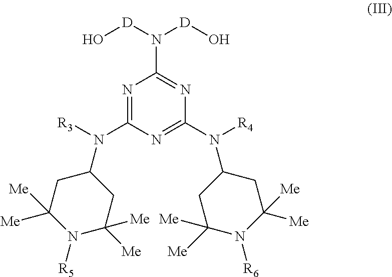

wherein:

D, R₃, R₄, R₅ and R₆ have the meanings above described in formula (II) and compounds of formula (IV)

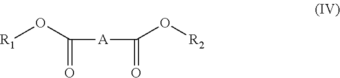

wherein:

R₁, R₂ and A have the meanings above described in formula (I).

The compounds of formula (IV) are used in stoichiometric excess to those of formula III, to ensure that the terminal groups of the polymer chains formed are the ester type described in formula (I). At the end of the polymerisation reaction, the excess of the unreacted compounds of formula (IV) is distilled off under vacuum.

Examples of compounds of formula (IV) are:
dimethyl esters, diethyl esters, dibutyl esters and dioctyl esters or mixed esters of linear dicarboxylic acids such as propanedioic, butanedioic, hexanedioic, octanedioic, decanedioic and dodecanedioic acids;
dimethyl esters, diethyl esters, dibutyl esters and dioctyl esters or mixed esters of aromatic dicarboxylic acids such as terephthalic acids and phthalic acid;
dimethyl esters, diethyl esters, dibutyl esters and dioctyl esters of cycloalkane dicarboxylic acids such as 1,2-dicarboxycyclohexane, 1,3-dicarboxycyclohexane and 1,4-dicarboxycyclohexane.

The intermediates of formula (III) can easily be synthesised by well-known methods by reacting cyanuryl chloride with an equivalent of each of the compounds of formulas (V), (VI) and (VII) as described, for example, in JPS58152881:

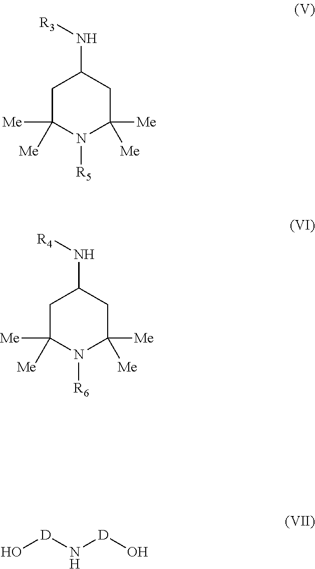

wherein D, R₃, R₄, R₅, R₆ and D have the meanings above described in formula II.

The synthesis conditions of the compounds of formula (I) are those typical of the polycondensation reactions used to prepare polyesters. A typical procedure for the preparation of linear polyesters consists of polycondensation of diols with dicarboxylic acids in the presence of suitable catalysts, with the removal of the water released during the esterification reaction. Similarly, linear polyesters can also be obtained by polycondensation of diols and diesters of dicarboxylic acids in the presence of suitable trans-esterification catalysts. In this case, the polymerisation reaction is conducted by removing the alcohols released during the reaction, which are generally low-boiling. The reaction is usually conducted at temperatures ranging between 50 and 300° C., preferably between 100 and 200° C., and at pressures ranging between 0 and 2 bars, preferably operating under vacuum.

The polyesters can be prepared in bulk or in the presence of suitable solvents inert to the esterification reaction. Examples of suitable solvents are saturated or aromatic hydrocarbons such as heptane, decane, toluene and xylene, ketones such as cyclohexanone and methyl-isobutylketone, and nitriles such as benzonitrile. Typical catalysts can be acids such as sulphuric acid, methanesulphonic acid and p-toluenesulphonic acid, bases such as lithium amide and sodium methylate, alkyl titanates such as tetraisopropyl titanate, and tin compounds such as dibutyltin oxide.

The polymerisation reactions are conducted with a well-determined stoichiometric excess of the compound of formula (IV) and with high reaction conversions, with the aim of obtaining polymer chains of a defined molecular weight terminating with ester groups at both ends.

The compounds of formula (I) are efficient light stabilisers for polymer materials, especially for polyolefins, either used alone or mixed with many other known stabilisers.

Mixtures of the compounds of formula (I) with other known stabilisers are also part of the invention.

In particular, mixtures of the compounds of formula (I) with at least one of the compounds of formula (P), (Q), (R), (S), (T) or (U) are preferred.

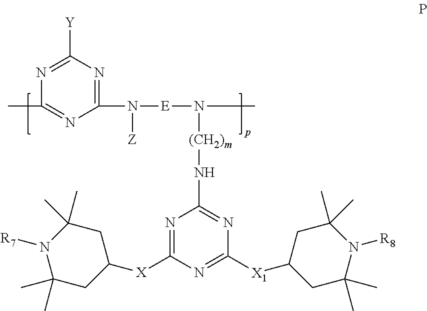

wherein p is between 3 and 20;

m is between 2 and 12;

R₇ and R₈, which may be the same or different, represent hydrogen, a straight or branched C₁-C₁₂ alkyl group, a C₃-C₈ alkenyl group or a C₇-C₁₉ aralkyl group;

X and X₁, which may be the same or different, represent oxygen or a group of formula (VIII)

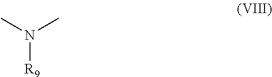

wherein R₉ is hydrogen, a straight or branched C₁-C₁₂ alkyl group, a C₅-C₁₂ cycloalkyl group or a C₇-C₁₂ aralkyl group;

E represents a —(CH₂)ₐ— group wherein a is between 2 and 12, on the proviso that a is different from m;

Z represents a C₁-C₁₈ alkyl group or a group of formula (IX)

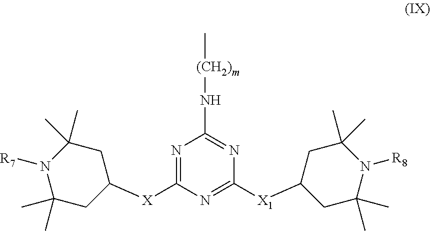

wherein m, X, X₁, R₇ and R₈ are as defined above,

or a group of formula (X)

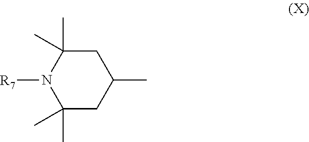

wherein R7 is as defined above;

Y represents the O—R₁₁ and S—R₁₁ groups or a group of formula (XI)

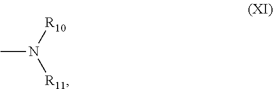

wherein R₁₀ and R₁₁, which may be the same or different, are hydrogen, a straight or branched C₁-C₁₈ alkyl group, a C₅-C₁₂, cycloalkyl group, a C₇-C₁₂ aralkyl group or a C₆-C₁₂ aryl group, or can form, together with the nitrogen atom to which they are bonded, a morpholino group or a C₅-C₇ heterocycle;

and the piperidino group (XII)

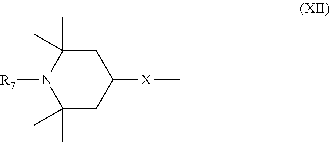

wherein R₇ and X are as defined above;

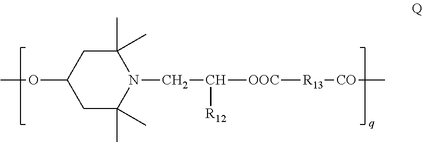

wherein

R₁₂ is hydrogen or methyl;

R₁₃ is a direct bond or a C₁-C₁₀ alkylene group;

q is an integer ranging from 2 to 50;

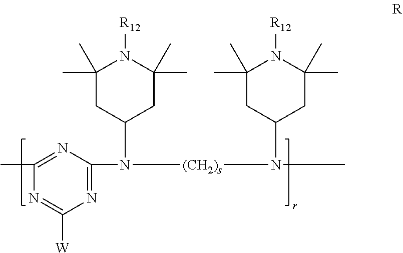

wherein:

r is an integer ranging from 2 to 50

s is an integer ranging from 2 to 10 (nella figura c'é (CH₂)₈ ma deve essere (CH2)s)

R₁₂ is as defined above for the compounds of Formula Q;

W is a group of formula (XIII), (XIV) or (XV):

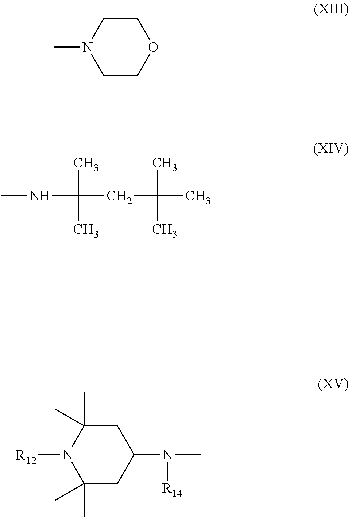

wherein:

R₁₄ is a straight or branched C₁-C₄ alkyl group,

R₁₂ is as defined above for the compounds of Formula R;

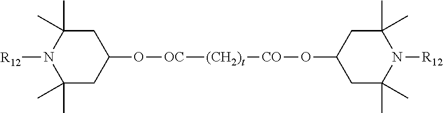

wherein:

t is an integer ranging from 2 to 10;

R₁₂ is as defined above for the compounds of Formula R;

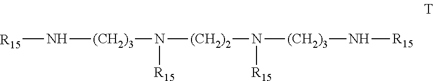

wherein:

R₁₅ represents the group of formula (XVI)

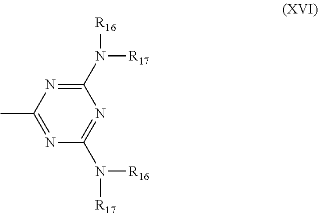

wherein R₁₆ and R_{17,} independently of one another, are selected from the group formed by hydrogen, straight or branched C₁-C₄ alkyl groups and the group of formula (XVII)

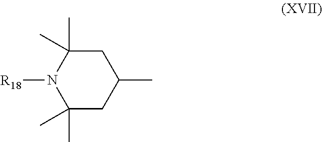

wherein R₁₈ is hydrogen, a straight or branched C₁-C₄ alkyl group or an OR₁₉ group wherein R₁₉ is hydrogen or a straight or branched C₁-C₈ alkyl group;

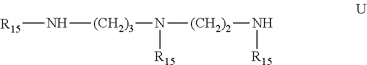

wherein R15 has the meanings defined above for the compounds of formula T.

Said mixtures preferably contain 10% to 90% by weight of the compound of formula (I). More preferably, said mixtures contain 25% to 75% by weight of the compound of formula (I).

Most preferably, said mixtures contain 40% to 60% by weight of the compound of formula (I).

It has been observed that mixtures containing the compounds of formula (I), in particular with n=3-20, R₄=n-butyl and A=—(CH₂)₈—, give polymer materials better stability against photodegradation and the oxidative action of air.

The compounds of formulas P, Q, R, S, T and U and their preparations are known and described in U.S. Pat. No. 4,477,615, EP2,632,914, U.S. Pat. Nos. 3,840,494, 3,640,928, 4,331,586, EP93693, U.S. Pat. No. 4,263,434, JP57038589 and U.S. Pat. No. 6,046,304.

An example of a compound of formula P which can be used in the mixtures according to the invention is the product commercially known by the name of Uvasorb HA88 (3V Sigma S.p.A.).

Another example of a compound of formula P which can be used in the mixtures according to the invention is the product commercially known by the name of Uvasorb HA10 (3V Sigma S.p.A.).

An example of a compound of formula Q which can be used in the mixtures according to the invention is the product commercially known by the name of Uvasorb HA22 (CAS RN=65447-77-0).

An example of a compound of formula R, wherein W=residue of formula (XIII), which can be used in the mixtures according to the invention is the product commercially known by the name of Cyasorb UV-3346 (CAS RN=82451-48-7).

An example of a compound of formula R, wherein W=residue of formula (XIV), which can be used in the mixtures according to the invention is the product commercially known by the name of Chimassorb 944 (CAS RN=71878-19-8).

An example of a compound of formula R, wherein W=residue of formula (XV), which can be used in the mixtures according to the invention is the product commercially known by the name of Chimassorb 2020 (CAS RN=192268-64-7).

An example of a compound of formula S, wherein R₁₂=H and t=8, which can be used in the mixtures according to the invention is the product commercially known by the name of Uvasorb HA77 (CAS RN=52829-07-9).

An example of a compound of formula S, wherein R₁₂=methyl and t=8, which can be used in the mixtures according to the invention is the product commercially known by the name of Uvasorb HA29 (CAS RN=41556-26-7).

An example of a compound of formula T, wherein R₁₆=n-butyl and R₁₇=residue of formula (XVII) having R₁₈=methyl, which can be used in the mixtures according to the invention is the product commercially known by the name of Chimassorb 119 (CAS RN=106990-43-6).

The mixtures of the invention can be obtained by any known method, for example (a) by melting the compounds of formula (I) together with one or more of the compounds of formula P, Q, R, S, T, and/or U, and then grinding or granulating the mixture obtained, (b) dissolving the ingredients in a common solvent and evaporating the solution until dry, or (c) incorporating the compounds separately in the polymer substrate to be stabilised, thereby obtaining the mixture in situ.

A further subject of the invention is the use of the compounds of formula (I) alone, mixed together or mixed with at least one of the compounds of formula P, Q, R, S, T or U as stabilisers for polymers, in particular for polyolefin polymers.

According to the invention, the polymers comprise polyethylene, polypropylene, polystyrene, polybutadiene, polyisoprene and the copolymers thereof, polyvinyl chloride, polivinylidene chloride and the copolymers thereof, polyvinyl acetate and the copolymers thereof, in particular with ethylene; polyesters such as polyethylene terephthalate; polyamides such as Nylon 6 or 6,6; and polyurethanes.

The compounds of the invention and the mixtures of the invention can be incorporated in the polymers by any known method for mixing additives and polymer materials; for example by:
mixing with the polymer, which can be in the form of a powder or granulate, in a suitable mixer;
addition in the form of a solution or suspension in a suitable solvent, and subsequent removal of the solvent from the polymer, which may be in the form of a powder, granulate or suspension, after thorough mixing;
addition to the polymer during its preparation, for example during the last stage of preparation.

The mixtures of the invention can be added together with other types of stabilisers and additives generally used in the art, such as antioxidants based on phenols, amines or phosphites; UV radiation absorbers based on benzophenones or benzotriazoles; nickel-based stabilisers; plasticisers, lubricants, antistatic agents, flame retardants, corrosion inhibitors, metal deactivators and mineral fillers such as titanium dioxide, aluminium oxide and the like.

Examples of said additives are:

1. Antioxidants

1.1. Alkylated phenols, such as: 2,6-di-tert-butyl-4-methylphenol; 2-tert-butyl-4,6-di-methylphenol; 2,6-di-tert-butyl-4-ethylphenol; 2,6-di-tert-butyl-4-butylphenol; 2,6-di-tert-butyl-4-isobutylphenol; 2,6-di-cyclopentyl-4-methylphenol; 2-(α-methylcyclohexyl)-4,6-di-methylphenol; 2,6-di-octadecyl-4-methylphenol; 2,4,6-tricyclohexylphenol; 2,6-di-tert-butyl-4-methoxymethylphenol; straight and branched nonylphenols, such as 2,6-di-nonyl -4-methylphenol; 2,4-di-methyl-6-(1′-methylundecyl)phenol; 2,4-di-methyl-6-(1′-heptadecyl) -phenol and mixtures thereof.

1.2. Alkyl-thiomethylphenols, such as: 2,4-di-octyl-thiomethyl-6-tert-butylphenol; 2,4-di-octyl-thiomethyl-6-methylphenol; 2,4-di-octyl-thiomethyl-6-ethylphenol; 2,6-di-dodecylthiomethyl -4-nonylphenol.

1.3. Hydroquinones and alkylated hydroquinones, such as: 2,6-di-tert-butyl-4-methoxyphenol; 2,5-di-tert-butyl-hydroquinone; 2,5-di-tert-amyl-hydroquinone; 2,6-diphenyl-4-octadecyloxyphenol; 2, 6-di-tert-butyl-hydroquinone; 2,5-di-tert-butyl-4-hydroxyanisole; 3,5-di-tert-butyl-4-hydroxyanisole; 3,5-di-tert-butyl-4-hydroxyphenyl stearate; bis(3,5-di-tert-butyl-4-hydroxyphenyl)adipate.

1.4. Tocopherols, such as α-tocopherol; γ-tocopherol; β-tocopherol; δ-tocopherol and mixtures thereof (vitamin E).

1.5. Hydroxylated thiodiphenyl ethers, such as: 2,2′-thiobis(6-tert-butyl-4-methylphenol); 2,2′-thiobis(4-octylphenol); 4,4′-thiobis(6-tert-butyl-3-methylphenol); 4,4 ′-thiobis(6-tert-butyl-2-methylphenol); 4,4′-bis(2, 6-di-methyl-4-hydroxyphenyl)-disulphide.

1.6. Alkylidene bisphenols, such as: 2,2′-methylenebis(6-tert-butyl-4-methylphenol); 2,2′-methylenebis(6-tert-butyl-4-ethylphenol); 2,2′ -methylenebis(4-methyl-6-(α-methylcyclohexyl)phenol); 2,2′-methylenebis(4-methyl-6-cyclohexylphenol); 2,2′-methylenebis(6-nonyl-4-methylphenol); 2,2′-methylenebis-(4,6-di-tert-butylphenol); 2,2′-ethylidenebis-(4,6-di-tert-butylphenol); 2,2′-ethylidenebis(6-tert-butyl-4-isobutylphenol); 2,2′-methylenebis(6-(α-methylbenzyl)-4-nonylphenol); 2,2 ′-methylenebis(6-(α-adimethylbenzyl)-4-nonylphenol); 4,4′-methylenebis-(2,6-di-tert-butylphenol); 4,4′-methylenebis(6-tert-butyl-2-methyl-phenol); 1, 1-bis(5-tert-butyl-4-hydroxy-2-methylphenyl)-butane; 2, 6-bis(3-tert-butyl-5-methyl-2-hydroxybenzyl)-4-methylphenol; 1, 1,3 -tris-(5-tert-butyl-4-hydroxy-2-methylphenyl)-butane; 1, 1-bis(5-tert-butyl -4-hydroxy-2-methylphenyl)-3-n-dodecyl-mercaptobutane; ethylene glycol bis(3,3-bis (3′-tert-butyl-4′-hydroxyphenyl)-butyrate); bis(2-(3′-tert-butyl-2′-hydroxy-5′-methylbenzyl) -6-tert-butyl-4-methylphenyl)-terephthalate; bis(3-tert-butyl-4-hydroxy-5-methylphenyl) -dicyclopentadiene; 1,1-bis-(3,5-dimethyl-2-hydroxyphenyl)butane; 2,2,bis-(3,5-di-tert-butyl-4-hydroxyphenyl)propane; 2,2-bis(5 -tert-butyl-4-hydroxy-2-methylphenyl) -4-n-dodecyl-mercaptobutane; 1, 1,5,5 -tetra-(5 -tert-butyl-4-hydroxy-2-methylphenyl)-pentane.

1.7. O—, N— and S-benzyl derivatives, such as: 3,5,3′,5′-tetra-tert-butyl-4-4′-dihydroxydibenzyl ether; octadecyl-4-hydroxy-3,5-dimethylbenzyl-mercapto acetate; tridecyl-4-hydroxy-3,5-di-tert-butyl-benzylmercapto acetate; tri(3,5-di-tert-butyl-4-hydroxybenzyl)amine; bis(4-tert-butyl-3 -hydroxy-2,6-dimethylbenzyl)dithioterephthalate; bis(3,5-di-tert-butyl-4-hydroxybenzyl)disulphide; isooctyl-3,5-di-tert-butyl-4-hydroxybenzylmercaptoacetate.

1.8. Malonates containing the hydroxybenzyl group, such as: dioctadecyl-2,2-bis-(3,5-di-tert-butyl-2-hydroxybenzyl)malonate; dioctadecyl-2-(3-tert-butyl-4-hydroxy-5-methylbenzyl) malonate; di-dodecylmercaptoethyl-2,2-bis-(3,5-di-tert-butyl-4-hydroxybenzyl)malonate; bis(4-(1, 1,3,3-tetramethylbutyl)-phenyl)-2,2-bis-(3,5-di-tert-butyl -4-hydroxybenzyl)malonate.

1.9. Aromatic hydroxybenzyl compounds, such as: 1,3,5-tris-(3,5-di-tert-butyl-4-hydroxybenzyl) -2,4,6-trimethylbenzene; 1,4-bis-(3,5-di-tert-butyl-4-hydroxybenzyl)-2,3,5,6-tetramethylbenzene; 2,4,6-tris-(3,5-di-tert-butyl-4-hydroxybenzyl)-phenol.

1.10. Triazine derivatives, such as: 2,4-bis(octylmercapto)-6-(3,5-di-tert-butyl-4-hydroxyanilino) -1,3,5-triazine; 2-octylmercapto-4-6-bis(3,5-di-tert-butyl-4-hydroxyanilino) -1,3,5-triazine; 2-octylmercapto-4-6-bis(3,5-di-tert-butyl-4-hydroxyphenoxy) -1,3,5-triazine; 2,4, 6-tris(3,5-di-tert-butyl-4-hydroxy-phenoxy)-1,3,5-triazine; 1,3,5 -tris-(3,5-di-tert-butyl-4-hydroxybenzyl)-isocyanurate; 1,3,5 -tris-(4-tert-butyl -3-hydroxy-2,6-dimethylbenzyl)isocyanurate; 2,4,6-tris(3,5-di-tert-butyl-4-hydroxyphenylethyl) -1,3,5-triazine; 1,3,5-tris-(3,5-di-tert-butyl-4-hydroxyphenylpropionyl) -hexahydro-1,3,5-triazine; 1,3,5-tris-(3,5-dicyclohexyl-4-hydroxybenzyl)isocyanurate.

1.11. Benzylphosphonates, such as: dimethyl-2,5 -di-tert-butyl-4-hydroxybenzylphosphonate; diethyl-3,5-di-tert-butyl-4-hydroxybenzylphosphonate; dioctadecyl-3,5-di-tert-butyl-4-hydroxybenzylphosphonate; dioctadecyl-5-tert-butyl-4-hydroxy -3-methylbenzylphosphonate; the calcium salt of 3,5-di-tert-butyl-4-hydroxybenzylphosphonic acid monoethyl ester.

1.12. Acylaminophenols, such as lauric acid 4-hydroxyanilide, stearic acid 4-hydroxyanilide and octyl N-(3,5-di-tert-butyl-4-hydroxyphenyl)carbamate.

1.13. Esters of β-(3,5-di-tert-butyl-4-hydroxyphenyl)propionic acid with mono- or polyhydric alcohols, such as: methanol, ethanol, n-octanol, iso-octanol and octadecanol; 1,6-hexanediol; 1,9-nonanediol; ethylene glycol; 1,2-propanediol; neopentyl glycol; thiodiethylene glycol; diethylene glycol; triethylene glycol; pentaerythritol; tri-(hydroxyethyl)isocyanurate; N,N′-bis(hydroxyethyl)oxamide; 3-thioundecanol; 3-thiopentadecanol; trimethylhexanediol; trimethylolpropane; 4-hydroxymethyl-1-phospho -2,6,7-trioxabicyclo(2,2,2)octane.

1.14. Esters of β-(5-tert-butyl-4-hydroxy-3-methylphenyl)propionic acid with mono- or polyhydric alcohols, such as: methanol, ethanol, n-octanol, iso-octanol and octadecanol; 1,6-hexanediol; 1,9-nonanediol; ethylene glycol; 1,2-propanediol; neopentyl glycol; thiodiethylene glycol; diethylene glycol; triethylene glycol; pentaerythritol; tri-(hydroxyethyl)isocyanurate; N,N′-bis(hydroxyethyl)oxamide; 3-thioundecanol; 3-thiopentadecanol; trimethylhexanediol; trimethylolpropane; 4-hydroxymethyl-1-phospho -2,6,7-trioxabicyclo(2,2,2)octane.

1.15. Esters of β-)3,5-dicyclohexyl-4-hydroxyphenyl)propionic acid with mono- or polyhydric alcohols, such as: methanol, ethanol, n-octanol, iso-octanol and octadecanol; 1,6-hexanediol; 1,9-nonanediol; ethylene glycol; 1,2-propanediol; neopentyl glycol; thiodiethylene glycol; diethylene glycol; triethylene glycol; pentaerythritol; tri-(hydroxyethyl)isocyanurate; N,N′-bis(hydroxyethyl)oxamide; 3-thioundecanol; 3-thiopentadecanol; trimethylhexanediol; trimethylolpropane; 4-hydroxymethyl-1-phospho -2,6,7-trioxabicyclo(2,2,2)octane.

1.16. Esters of 3,5-di-tert-butyl-4-hydroxyphenyl acetic acid with mono- or polyhydric alcohols, such as: methanol, ethanol, n-octanol, iso-octanol and octadecanol; 1,6-hexanediol; 1,9-nonanediol; ethylene glycol; 1,2-propanediol; neopentyl glycol; thiodiethylene glycol; diethylene glycol; triethylene glycol; pentaerythritol; tri-(hydroxyethyl)isocyanurate; N,N′-bis(hydroxyethyl)oxamide; 3-thioundecanol; 3-thiopentadecanol; trimethylhexanediol; trimethylolpropane; 4-hydroxymethyl-1-phospho -2,6,7-trioxabicyclo(2,2,2)octane.

1.17. Amides of β-(3,5-di-tert-butyl-4-hydroxyphenyl)propionic acid, such as: N,N′-bis-(3,5-di-tert-butyl-4-hydroxyphenylpropionyl)-hexamethylenediamide; N,N′-bis-(3,5-di-tert-butyl-4-hydroxyphenylpropionyl)-trimethylenediamide; N,N′-bis-(3,5-di-tert-butyl -4-hydroxyphenylpropionyl)-hydrazide; N,N′-bis-(2-(3-(3,5-di-tert-butyl-4-hydroxyphenyl) propionyloxy)ethyl)-oxamide.

1.18. Ascorbic acid (vitamin C). 1.19. Amine antioxidants, such as: N,N′-di-isopropyl-p-phenylenediamine; N,N′-di-sec-butyl-p-phenylenediamine; N,N′-bis(1,4-dimethylpentyl)-p-phenylenediamine; N,N′-bis(1-ethyl-3-methylpentyl)-p-phenylenediamine; N,N′-bis(1-methylheptyl) -p-phenylenediamine; N,N′-dicyclohexyl-p-phenylenediamine; N,N′-diphenyl-p-phenylenediamine; N,N′-bis-(2-naphthyl)-p-phenylenediamine; N-isopropyl-N′-phenyl-p-phenylenediamine; N-(1,3-dimethylbutyl)-N′-phenyl-p-phenylenediamine; N-(1-methylheptyl)-N′-phenyl-p-phenylenediamine; N-cyclohexyl-N′-phenyl-p-phenylenediamine, 4-(p-toluen-sulphamoyl)diphenylamine; N,N′-dimethyl-N,N′-di-sec-butyl-p-phenylenediamine; diphenylamine; N-allyl-diphenylamine; 4-isopropoxydiphenylamine, N-phenyl-1-naphthylamine; N-(4-tert-octylphenyl)-1-naphthylamine; N-phenyl-2-naphthylamine; p,p′-di-tert-octyl-diphenylamine; 4-n-butyl-aminophenol; 4-butyryl-aminophenol; 4-nonanoyl-aminophenol; 4-dodecanoyl-aminophenol; 4-octadecanoyl-aminophenol; bis(4-methoxyphenyl)amine; 2,6-di-tert-butyl -4-dimethylaminomethylphenol; 2,4′-diaminodiphenylmethane; 4,4′-diaminodiphenyl-methane; N,N,N′,N′-tetramethyl-4,4′-diaminodiphenylmethane; 1,2-bis-((2-methylphenyl)-amino)ethane; 1,2-bis-(phenylamine)propane; o-tolyl-biguanide; bis-(4-(1′,3′-dimethylbutyl)phenyl)amine; tert-octyl-N-phenyl -1-naphthylamine; mixtures of dialkylated tert-butyl/tert-octyl-diphenylamines; mixtures of mono- and di-alkyl nonyldiphenylamines; mixtures of mono- and di-alkyl dodecyldiphenylamines; mixtures of mono- and di-alkyl isopropyl/isohexyldiphenylamines; mixtures of mono- and di-alkyl tert-butyldiphenylamines; 2,3 -dihydro-3,3-dimethyl-4H-1,4-benzothiazine; phenothiazine; mixtures of mono- and dialkyl tert-butyl/tert-octylphenothiazines, mixtures of mono- and dialkyl tert-octyl-phenothiazines; N-allylphenothiazine; N,N,N′,N′-tetraphenyl-1,4-diamino-2-butene; N,N′-bis-(2,2,6,6-tetramethyl-piperidyl-4-hexamethylenediamine; bis(2,2,6,6-tetramethylpiperid-4-yl)sebacate; 2,2,6,6-tetramethylpiperid -4-one; 2,2,6,6-tetramethylpiperid-4-ol.

2. UV radiation absorbers and light stabilisers

2.1. 2-(2′-hydroxyphenyl)benzotriazoles, such as: 2-(2′-hydroxy-5-methylphenyl)benzotriazole; 2-(3′,5′-di-tert-butyl-2′-hydroxyphenyl)benzotriazole; 2-(5′-tert-butyl -2′-hydroxyphenyl)benzotriazole; 2 -(2′-hydroxy-5′-(1,1,3,3 -tetramethylbutyl)-enyl) benzotriazole; 2-(3′,5′-di-tert-butyl-2′-hydroxyphenyl)-5-chloro-benzotriazole; 2-(3′-tert-butyl-2′-hydroxy-5′-methylphenyl)-5-chloro-benzotriazole; 2-(3′-sec-butyl-5′-tert -butyl-2′-hydroxyphenyl)-benzotriazole; 2-(2′-hydroxy-4′-octyloxyphenyl) benzotriazole; 2-(3′,5′-di-tert-amyl-2′-hydroxyphenyl)benzotriazole; 2-(3′,5′-bis-(α,α-dimethylbenzyl)-2′-hydroxyphenyl)benzotriazole; 2-(3′-tert-butyl-5′-(2-(2-ethylhexyloxy)-carbonylethyl)-2′-hydroxyphenyl)-5-chloro-benzotriazole; 2-(3′-tert-butyl -2′-hydroxy-5′-(2-octyloxycarbonylethyl)phenyl)-5-chloro-benzotriazole; 2-(3′-tert-butyl -2′-hydroxy-5′-(2-methoxycarbonylethyl)phenyl)-5-chloro-benzotriazole; 2-(3′-tert-butyl -2′-hydroxy-5′-(2-methoxycarbonylethyl)phenyl)-benzotriazole; 2-(3′-tert- butyl-2′-hydroxy-5′-(2-octyloxycarbonylethyl)phenyl)-benzotriazole; 2-(3′-tert-butyl-5′-(2-(2-ethylhexyloxy)-carbonylethyl)-2′-hydroxyphenyl)-benzotriazole; 2-(3′-dodecyl-2′-hydroxy -5′-methylphenyl)-benzotriazole; 2-(3′-tert-butyl-2′-hydroxy-5′-(2-isooctyloxy-carbonylethyl) -phenylbenzotriazole; 2,2′-methylene-bis-(4-(1,1,3,3-tetramethylbutyl)-6-benzotriazol -2-ylphenol); the product of transesterification of 2-(3′-tert-butyl-5′-(2-methoxycarbonylethyl) -2′-hydroxyphenyl)-2H-benzotriazole with polyethylene glycol 300; (R—CH₂—CH₂—COO—CH₂—CH₂—)₂— wherein R can be: 3′-tert-butyl-4′-hydroxy-5′-2H-benzotriazol -2-ylphenyl; 2-(2′-hydroxy-3′-(α,α-dimethylbenzyl)-5′-(1, 1,3,3 -tetramethylbutyl) -phenyl)benzotriazole; 2-(2′-hydroxy-3′-(1,1,3,3 -tetramethylbutyl)-5′-(α,α-dimethylbenzyl) phenyl)benzotriazole.

2.2. 2-hydroxybenzophenones, such as the 4-hydroxy; 4-methoxy; 4-octyloxy; 4-decyloxy; 4-dodecyloxy; 4-benzyloxy; 4,2′,4′-tri-hydroxy and 2′-hydroxy-4,4′-dimethoxy derivatives.

2.3. Esters of substituted and non-substituted benzoic acids, such as: 4-tertbutyl-phenyl-salicylate; phenyl salicylate; octylphenyl salicylate; dibenzoyl resorcinol; bis-(4-tert-butyl-benzoyl) -resorcinol; benzoyl resorcinol; 2,4-di-tert-butylphenyl 3,5-di-tert-butyl -4-hydroxybenzoate; hexadecyl 3,5-di-tert-butyl-4-hydroxybenzoate; octadecyl 3,5-di-tert-butyl-4-hydroxy-benzoate; 2-methyl-4, 6-di-tert-butylphenyl 3,5-di-tert-butyl-4-hydroxybenzoate:

2.4. Acrylates, such as: ethyl α-cyano-β,β-diphenylacrylate; isooctyl α-cyano-β,β-diphenylacrylate; methyl α-carbomethoxycinnamate; methyl α-cyano-β-methyl-p-methoxycinnamate; butyl α-cyano-β-methyl-p-methoxy-cinnamate; methyl α-carbomethoxy-p-methoxycinnamate and N-(β-carbomethoxy-(β-cyanovinyl)-2-methylindoline.

2.5. Nickel derivatives, such as: 1:1 or 1:2 complexes of nickel with 2,2′-thio-bis-(4-(1,1,3,3-tetramethylbutyl)phenol, with or without binders such as n-butylamine, triethanolamine or N-cyclohexyldiethanolamine; nickel dibutylthiodicarbamate; nickel salts of 4-hydroxy-3,5-di-tert-butylbenzylphosphonic acid mono-alkyl esters (such as methyl or ethyl esters); nickel complexes with keto-oxime, for example with 2-hydroxy-4-methylphenyl undecyl-keto-oxime; nickel complexes with 1-phenyl-4-lauroyl-5-hydroxy-pyrazole, with or without additional binders.

2.6. Oxamides, such as: 4,4′-dioctyloxy-oxalanilide; 2,2′-diethoxy-oxalanilide; 2,2′-dioctyloxy-5,5′-di-tert-butyl-oxalanilide; 2,2′-didodecyloxy-5,5′-di -tert-butyl -oxalanilide; 2-ethoxy-2′-ethyloxy-oxalanilide; N,N′-bis(3-dimethylaminopropyl)-oxalanilide; 2-ethoxy-2′-ethyl-5,4′-di-tert-butyl-oxalanilide; mixtures of disubstituted o- and p-methoxy oxalanilides and mixtures of disubstituted o- and p-ethoxy oxalanilides.

2.7. 2-(2-hydroxyphenyl)-1,3,5-triazines, such as: 2,4,6-tris(2-hydroxy-4-octyloxyphenyl) -1,3,5-triazine; 2-(2-hydroxy-4-octyloxyphenyl)-4,6-bis(2,4-dimethylphenyl) -1,3,5-triazine; 2,4-bis(2-hydroxy-4-propyloxyphenyl)-6-(2,4-dimethylphenyl) -1,3,5-triazine; 2-(2-hydroxy-4-octyloxyphenyl)-4,6-bis-(4-methyl-phenyl) -1,3,5-triazine; 2-(2-hydroxy-4-dodecyloxyphenyl)-4,6-bis-(2-4-dimethyl-phenyl) -1,3,5-triazine; 2-(2-hydroxy-4-tridecyloxyphenyl)-4,6-bis-(2-4-dimethyl -phenyl)-1,3,5 -triazine; 2-(2-hydroxy-4-(2-hydroxy-3-butyloxy-propoxy)phenyl)-4,6-bis(2,4-dimethyl) -1,3,5 -triazine; 2-(2-hydroxy-4-(2-hydroxy-3-octyloxy-propoxy)-phenyl)-4, 6-bis (2,4-dimethyl)-1,3,5-triazine; 2-(4-(dodecyloxy/tridecyloxy-2-hydroxypropoxy)-2-hydroxyphenyl) -4,6-bis (2,4-dimethylphenyl)-1,3,5-triazine; 2-(2-hydroxy-4-(2-hydroxy-3-dodecyloxy-propoxy)phenyl)-4,6-bis(2,4-dimethylphenyl)-1,3,5-triazine; 2-(2-hydroxy-4-hexyloxy)phenyl-4,6-diphenyl -1,3,5 -triazine; 2-(2-hydroxy-4-methoxyphenyl)-4,6-diphenyl -1,3,5-triazine; 2,4,6-tris(2-hydroxy-4-(3-butoxy-2-hydroxy-propoxy)phenyl -1,3,5-triazine; 2-(2-hydroxyphenyl)-4-(4-methoxyphenyl)-6-phenyl-1,3,5-triazine; 2-(2-hydroxy-4-(3-(2-ethylhexyl-1-oxy)-2-hydroxypropyloxy)phenyl)-4,6-bis(2,4-dimethylphenyl) -1,3,5-triazine.

2.8. Triazine derivatives, such as: diethylhexyl butamido triazone, ethylhexyl triazone, 2,4-bis-[4-[5-(1, 1-dimethyl-propyl)benzoxazol-2-yl]phenylimino]-6-[(2-ethylhexyl)imino ]-1,3,5-triazine, tris-biphenyltriazine, bis-ethylhexyloxyphenol-methoxyphenyl-triazine. 3. Metal deactivators, such as: N,N′-diphenyloxamide; N-salicylal-N′-salicylol-hydrazine; N,N′-bis(salicylol)hydrazine; N,N′-bis-(3,5-di-tert-butyl-4-hydroxyphenylpropionyl) -hydrazine; 3-salicylolamino-1,2,4-triazole; bis(benzylidene)-oxalyl dihydrazide; oxalanilide; isophthaloyl dihydrazide; sebacoyl bisphenylhydrazide; N,N′-diacetyladipoyl dihydrazide; N,N′-bis(salicyloyl)oxalyl dihydrazide; N,N′-bis(salicyloyl)thiopropionyl dihydrazide.

4. Phosphites and phosphonites, such as: triphenyl phosphite; diphenyl alkyl phosphites; phenyl dialkyl phosphites; tris(nonylphenyl)phosphite; trilauryl phosphite; trioctadecyl phosphite; distearyl pentaerythritol diphosphite; tris(2,4-di-tert-butyl-phenyl) phosphite; diisodecyl pentaerythritol diphosphite; bis(2,4-di-tert-butylphenyl) phosphite; diisodecyl pentaerythritol diphosphite; bis(2,4-di-tert-butylphenyl) pentaerythritol diphosphite; bis (2,6-di-tert-butyl-4-methylphenyl)-pentaerythritol diphosphite; diisodecyloxy-pentaerythritol diphosphite; bis-(2,4-di-tert-butyl-6-methylphenyl) pentaerythritol diphosphite; bis(2,4,6-tris(tert-butyl phenyl) pentaerythritol diphosphite; tristearyl sorbitol triphosphite; bis(2,4-di-tert-butyl-6-methylphenyl) methyl phosphite; bis(2,4-di-tert-butyl-6-methylphenyl) ethyl phosphite; 2,2′,2″-nitrilo(triethyltris(3,3′,5,5′-tetra-tert-butyl-1,1′-biphenyl-2,2′-idyl) phosphite); 2-ethylhexyl (3,3′,5,5′-tetra-tert-butyl-1,1′-biphenyl-2,2′-idyl) phosphite); tetra(2,4-di-tert-butylphenyl) 4-4′-biphenylene diphosphonite.

5. Hydroxylamines, such as: N,N-dibenzylhydroxylamine; N,N-diethylhydroxylamine; N,N-dioctylhydroxylamine; N,N-dilaurylhydroxylamine; N,N-ditetradecylhydroxylamine; N,N-dihexadecyl-hydroxylamine; N,N-dioctadecylhydroxylamine; N-hexadecyl-N-octadecylhydroxylamine; N-heptadecyl-N-octadecylhydroxylamine; N,N-dialkylhydroxylamines derived from hydrogenated tallow amines.

6. Nitrones, such as: N-benzyl-alpha-phenyl-nitrone; N-ethyl-alpha-methyl-nitrone; N-octyl-alpha-heptyl-nitrone; N-lauryl-alpha-undecyl-nitrone; N-tetradecyl-alpha -tridecyl-nitrone; N-hexadecyl-alpha-pentadecyl-nitrone; N-octadecyl-alpha-pentadecyl -nitrone; N-heptadecyl-alpha-heptadecyl-nitrone; N-octadecyl-alpha-hexadecyl -nitrone; nitrones derived from N,N-dialkylhydroxylamines obtained from hydrogenated tallow amines.

7. Thiosynergistic derivatives such as dilauryl thiodipropionate or stearyl thiodipropionate.

8. Antiperoxide agents, such as: esters of thiodipropionic acid with lauryl, stearyl, myristyl or tridecyl alcohols; mercaptobenzimidazole or 2-mercapto-benzoimidazole zinc salt; zinc dibutyldithiocarbamate; dioctadecyl disulphide; pentaerythritol tetrakis(β-dodecylmercapto) propionate.

9. Polyamide stabilisers, such as: copper salts in combination with iodides and/or phosphorated compounds and bivalent manganese salts.

10. Basic co-stabilisers, such as: melamine; polyvinylpyrrolidone; dicyandiamide; triallylcyanurate; urea derivatives; hydrazine derivatives; amines; polyamides; polyurethanes; alkali metal and alkaline earth metal salts of higher fatty acids such as calcium stearate and zinc stearate; magnesium behenate; magnesium stearate; sodium ricinoleate; potassium palmitate; pyrocatechol antimony or zinc salts.

11. Nucleating agents, such as: inorganic substances such as talc; metal oxides such as titanium dioxide or magnesium oxide; phosphates, carbonates or sulphates or alkali earth metal salts; organic compounds such as mono- or polycarboxylic acids and the salts thereof, such as 4-tert-butylbenzoic acid, adipic acid, diphenylacetic acid and sodium succinate; sodium benzoate; polymer compounds such as anionic copolymers.

12. Benzofuranones and indolinones, such as those described in U.S. Pat. Nos. 4,325,863; 4,338,244; 5,175,312; 5,216,052; 5,252,643; DE 4316611; DE 4316622; DE 4316876; EP 0589839; and EP 0591102; 3-(4-(2-acetoethoxy)phenyl)-5,7-di-tert-butyl -benzofuran-2-one; 5,7-di-tert-butyl-3 -(4-(2-stearoyloxyethoxy)phenyl)-benzofuran-2-one; 3,3′-bis(5,7-di-tert-butyl-3-(4-(2-hydroxyethoxy)phenyl)benzofuran-2-one); 5,7-di-tert-butyl -3-(4-ethoxyphenyl)-benzofuran-2-one; 3-(4-acetoxy-3,5-dimethylphenyl)-5,7-di-tert-butyl-benzofuran-2-one; 3-(3,5-dimethyl-4-pivaloyloxyphenyl)-5,7-di-tert-butyl -benzofuran-2-one; 3-(2,3-di-methylphenyl)-5,7-di-tert-butyl-benzofuran-2-one.

13. Fillers and reinforcing agents, such as: calcium carbonate; silicates; fibreglass; asbestos; talc; kaolin; mica; barium sulphate; metal oxides and hydroxides, titanium dioxide in its various forms, and carbon black; graphite; wood flour, fibre flour or other natural products; synthetic fibres.

14. Other additives, such as: plasticisers, lubricants, emulsifiers, pigments and rheology modifiers; catalysts; flow control agents; optical brighteners; flame retardants; antistatic agents and swelling agents.

The amount of mixtures according to the invention required for effective stabilisation of the polymer depends on a number of factors, such as the type and characteristics of the polymer, its intended use, the intensity of the radiation, and the duration of the likely exposure.

An amount of mixture ranging from 0.01 to 5% by weight of the polymer, preferably 0.1 to 1.0%, is usually sufficient.

The examples below illustrate the invention in detail.

### EXAMPLE 1: PREPARATION OF 2,2′-[[4,6-bis[butyl(2,2,6,6-tetramethyl-4-piperidinyl) amino]-1,3,5-triazin-2-yl]imino]bis-ethanol

corresponding to the molecule of formula III with R₃=R₄=butyl; R₅=R₆=H; D=—(CH₂)₂—

300 g of xylene, 2.13 g of sodium hydrogen carbonate and 55.5 g of cyanuryl chloride are loaded into a 1 L flask under nitrogen. 72.0 g of N-butyl-2,2,6,6-tetramethylpiperidin -4-amine and 97.4 g of 18.5% aqueous Na₂CO₃ are added to the stirred mixture in sequence, maintaining the temperature at between 50 and 80° C., followed by a further 55.5 g of N-butyl-2,2,6,6-tetramethylpiperidin-4-amine and 97.4 g of 18.5% aqueous Na₂CO₃. The reaction is completed by maintaining a slight reflux for two hours, and the lower aqueous phase is then discharged. Maintaining the temperature at about 80° C., 43.5 g of diethanolamine and 96.1 g of 18.5% aqueous Na₂CO₃ are added. The mixture is heated to reflux, removing the water with a Dean Stark apparatus. The mixture is kept under stirring at the same temperature for 6 h, and 150 g of xylene and 130 g of demineralised H₂O are then added. The reaction mixture is stirred at 90-95° C. for 30 minutes, and the underlying aqueous phase is then discharged. After a further aqueous wash, the solvent and residual water are removed under vacuum. 182.1 g of product is obtained by solidifying the molten mass. UPLC-MS analysis gave an assay value=99.69%.

### EXAMPLE 2: PREPARATION OF HALS1, CORRESPONDING TO A POLYMER OF FORMULA I WITH A=—(CH₂)₈—; R₁=R₂=methyl; B=a group of formula II with R₃=R₄=butyl; R₅=R₆=H; D=—(CH₂)₂— OBTAINABLE BY REACTING 2,2′-[[4,6-bis[butyl(2,2,6,6-tetramethyl-4-piperidinyl) -amino]-1,3,5-triazin-2-yl]imino]bis-ethanol WITH DECANEDIOIC ACID 1,10-dimethyl ester.

11.99 g (52.1 mmols) of dimethyl sebacate and 30.0 g (49.6 mmols) of triazine intermediate corresponding to the molecule of formula III in example 1 are loaded into a 250 mL flask under nitrogen. The temperature is increased to 140° C., and 0.05 g of titanium (IV) isopropoxide is added to the molten mass. The temperature is gradually increased to 160° C., and the reaction is completed by removing under vacuum the methanol formed and the excess unreacted dimethyl sebacate. 100 g of xylene is added and the catalyst is removed by hot aqueous washing, then azeotropically dried and clarified by hot filtration. After removal of the xylene under vacuum, the molten mass is cooled to obtain 34.6 g of the HALS1 product in the form of pale yellow flakes. A Mw=13849 g/mole was measured with GPC analysis.

### EXAMPLE 3: PREPARATION OF 2,2′-[[4,6-bis[butyl(1,2,2,6,6-pentamethyl-4-piperidinyl) amino]-1,3,5-triazin-2-yl]imino]bis-ethanol CORRESPONDING TO THE MOLECULE OF FORMULA III WITH R₃=R₄=butyl; R₅=R₆=methyl; D=—(CH₂)₂—

300 g of xylene, 2.13 g of NaHCO₃ and 55.5 g of cyanuryl chloride are loaded into a 1 L flask under nitrogen. 72.0 g of N-butyl-2,2,6,6-tetramethylpiperidin-4-amine and 97.4 g of 18.5% aqueous Na₂CO₃ are added to the stirred mixture in sequence, maintaining the temperature at between 50 and 80° C., followed by a further 55.5 g of N-butyl-2,2,6,6-tetramethylpiperidin-4-amine and 97.4 g of 18.5% aqueous Na₂CO₃. The reaction is completed by maintaining a slight reflux for two hours, and the lower aqueous phase is then discharged. Maintaining the temperature at about 80° C., 90.2 g of 30% aqueous formaldehyde and 46.1 g of 90% formic acid are added in about 2 hours. The reaction is completed at 85° C., then the mixture is cooled to 60° C. and 150 g of 10% aqueous NaOH is added. After mixing, the lower aqueous phase is discharged and the organic phase is washed with demineralised H₂O.

Maintaining the temperature at about 80° C., 42.8 g of diethanolamine and 96.1 g of 18.5% aqueous Na₂CO₃ are added. The mixture is heated to reflux, removing the water with a Dean Stark apparatus. The mixture is maintained under stirring at the same temperature for 6 h, then 130 g of demineralised H₂O is added. The reaction mixture is stirred at

90-95° C. for 30 minutes, and the underlying aqueous phase is then discharged. After a further aqueous wash, the solvent and residual water are removed under vacuum. 185.0 g of product is obtained by solidification of the molten mass. UPLC-MS analysis gave an assay value=99.52%

### EXAMPLE 4: PREPARATION OF HALS2, CORRESPONDING TO A POLYMER OF FORMULA I WITH A=—(CH₂)₈—; R1=R2=methyl; B=GROUP OF FORMULA II WITH R₃=R₄=butyl; R₅=R₆=methyl; D=—(CH₂)₂— OBTAINABLE BY REACTING 2,2′-[[4,6-bis[butyl(1,2,2,6,6-pentamethyl-4-piperidinyl) -amino]-1,3,5-triazin-2-yl]imino]bis-ethanol WITH DECANEDIOIC ACID 1,10-dimethyl ester.

11.49 g (49.9 mmols) of dimethyl sebacate and 30.07 g (47.5 mmols) of triazine intermediate corresponding to the molecule of formula III in example 3 are loaded into a 250 mL flask under nitrogen. The temperature is increased to 140° C. and 0.05 g of titanium (IV) isopropoxide is added to the molten mass. The temperature is gradually increased to 160° C., and the reaction is completed by removing under vacuum the methanol formed and the excess unreacted dimethyl sebacate. 100 g of xylene is added and the catalyst is removed by hot aqueous washing, then azeotropically dried and clarified by hot filtration. After removal of the xylene under vacuum, the molten mass is cooled to obtain 36.0 g of the HALS2 product in the form of pale yellow flakes.

A Mw=12178 g/mole was measured with GPC analysis.

Application examples

All amounts are expressed in weight unless otherwise stated.

### EXAMPLE 5: LIGHT STABILISATION OF POLYPROPYLENE FIBRE

4 samples were prepared by the following procedure: 1000 parts by weight of unstabilised polypropylene homopolymer (fluidity index about 10-12 g/10 min at 230° C. - 2.16 kP) were mixed in a laboratory mixer with 1.0 parts by weight of calcium stearate, 0.50 parts by weight of tris-(2,4-di-tert-butyl-phenyl) phosphite, 0.50 parts by weight of 1,3,5-tris-(3,5-di-tert-butyl-4-hydroxybenzyl) isocyanurate, to three of which 1.5 parts by weight of stabilisers HALS1, HALS2 and HALS3 respectively were added, wherein HALS3 is a hindered polyester polymer amine containing the hindered amino groups inserted directly into the polymer chain identified by CAS RN=65447-77-0.

The dry mixtures thus obtained were extruded in a laboratory extruder at 230° C., and granulated after cooling of the extrusion.

The granulates were then converted to a film with a thickness of 100 microns, using a laboratory press by compression moulding at 210° C.

Specimens obtained from the various films were exposed in a Weather-Ometer Mod. Ci35A, according to ISO 4892 (T black panel 63±2° C., dry cycle). Specimens were taken periodically to undergo a Carbonyl Index test, using the FT-IR spectrophotometry technique. The increase in the value of the Carbonyl Index provides an indication of the state of photooxidative degradation of the material, and is correlatable with the loss of mechanical properties resulting from the photooxidation of the material.

The parameter used to compare the light resistance of the samples was tom, defined as “exposure time in WOM, expressed in hours, on reaching a Carbonyl Index value of 0.10”.

The experimental results obtained are summarised in the Table.

TABLE
Light stability of 100 micron PP film

| Stabilisation | t_{0.10} |
|---|---|
| Without stabiliser | 700 |
| 0.15% HALS1 | 1730 |
| 0.15% HALS2 | 1800 |
| 0.15% HALS3 | 1250 |

The results of this example clearly demonstrate that the hindered polymer amines according to the present invention, containing the hindered amino groups in the mobile pendants of the polymer structure, are much more effective than those wherein the hindered amino group is inserted directly into the polymer chain.

1. Compounds of general formula (I): 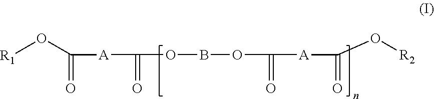
wherein:
n is an integer ranging from 2 to 100;
R₁ and R₂ are hydrogen or straight or branched, saturated or unsaturated C₁-C₁₈ alkyl groups or aromatic groups;
A is a C₁-C₂₀ alkylene or C₃-C₁₀ cycloalkylene group optionally containing one or more unsaturations, or an aromatic group;
and B is a group of formula (II): 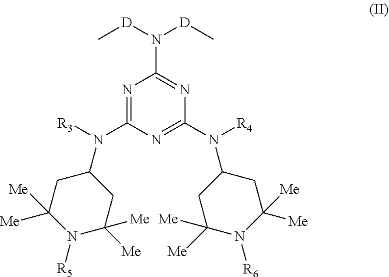
wherein:
D is an optionally unsaturated C₁-C₆ alkylene or isoalkylene group;
R₃, R₄, which can be the same or different, are hydrogen or C₁-C₈ alkyl;
R₅, R₆, which can be the same or different, are hydrogen or straight or branched C₁-C₄ alkyl or an -OG group wherein
G is hydrogen or straight or branched, saturated or unsaturated C₁-C₁₀ alkyl.

2. Compounds according to claim 1
wherein:
n is an integer ranging from 2 to 100;
R₁ and R₂ are hydrogen or C₁-C₄ alkyl;
A is a C₁-C₂₀ alkylene or C₃-C₁₀ cycloalkylene group optionally containing one or more unsaturations;
and B is a group of formula (II): 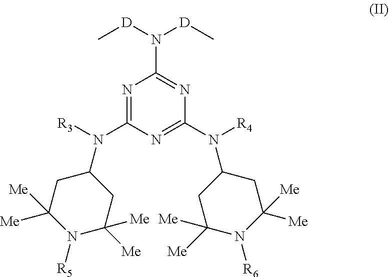
wherein:
D is a C₁-C₆ alkylene or isoalkylene group;
R₃, R₄, which can be the same or different, are hydrogen or C₁-C₈ alkyl;
R₅, R₆, which can be the same or different, are hydrogen or C₁-C₂ alkyl.

3. Compounds according to claim 1 wherein:
n ranges from 3 to 20,
A is a straight C₁-C₂₀ alkylene group,
D is a —CH₂—CH₂—, —CH₂—CH₂—CH₂—, —CH(CH₃)—CH₂— or —CH₂—CH(CH₃)— group,
R₁ and R₂ are methyl or ethyl,
R₃ and R₄ are hydrogen or n-butyl,
R₅ and R₆ are hydrogen or methyl.

4. Compounds according to claim 1
wherein:
n ranges from 3 to 20,
A is a —(CH₂)₈— group
D is a —CH₂—CH₂—, —CH(CH₃)—CH₂— or —CH₂—CH(CH₃)— group,
R₁ and R₂ are methyl,
R₃ and R₄ are n-butyl,
R₅ and R₆ are hydrogen or methyl.

5. Mixtures of compounds according to claim 1.

6. Mixtures of compounds according to claim 1, and at least one of the compounds of formulas (P), (Q), (R), (S), (T), (U) 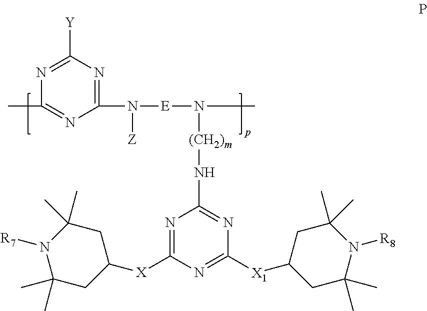
wherein p ranges from 3 to 20;
m ranges from 2 to 12;
R₇ and R_{8,} which can be the same or different, are hydrogen, a straight or branched C₁-C₁₂ alkyl group, a C₃-C₈ alkenyl group or a C₇-C₁₉ aralkyl group;
X and X₁, which can be the same or different, are oxygen or a group of formula (VIII) 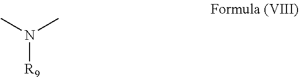
wherein R₉ is hydrogen, a straight or branched C₁-C₁₂ alkyl group, a C₅-C₁₂ cycloalkyl group or a C₇-C₁₂ aralkyl group;
E is a —(CH₂)ₐ— group wherein a ranges from 2 to 12, with the proviso that a is different from m;
Z is a C₁-C₁₈ alkyl group or a group of formula (IX) 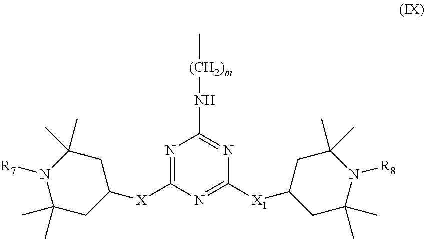
wherein m, X, X₁, R₇ and R₈ are as defined above,
or a group of formula (X) 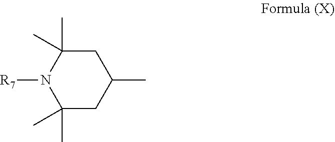
wherein R₇ is as defined above;
Y is an O—R₁₁ or S—R₁₁ group or a group of formula (XI) 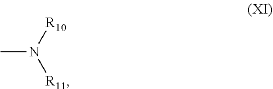
wherein R₁₀ and R₁₁, which can be the same or different, can be hydrogen, a straight or branched C₁-C₁₈ alkyl group, a C₅-C₁₂ cycloalkyl group, a C₇-C₁₂ aralkyl group or a C₆-C₁₂ aryl group or can form, together with the nitrogen atom they are linked to , a morpholino or C₅-C₇ heterocycle group;
and the piperidino group (XII) 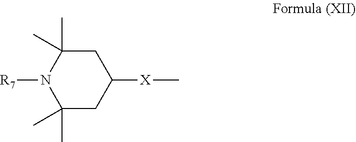
wherein R₇ and X are as defined above; 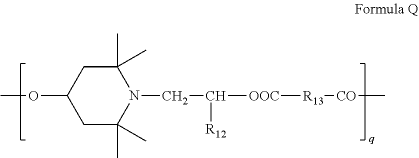
wherein
R₁₂ is selected from the group formed by hydrogen and methyl;
R₁₃ is a direct bond or a C₁-C₁₀ alkylene group;
q is an integer ranging from 2 to 50; 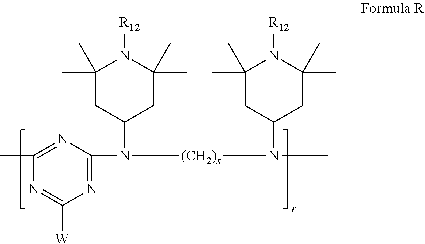
wherein:
r is an integer ranging from 2 to 50;
s is an integer ranging from 2 to 10;
R₁₂ is as defined above for the compounds of formula Q;
W is selected from the following groups of formulas (XIII), (XIV) and (XV): 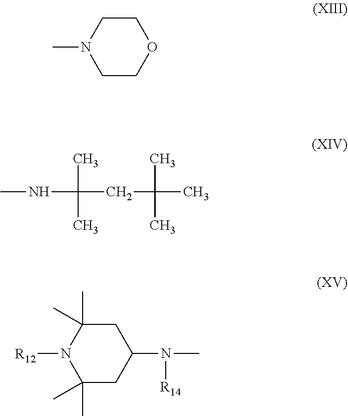
wherein:
R₁₄ is selected from the group formed by straight or branched C₁-C₄ alkyl groups;
R₁₂ is as defined above for the compounds of formula R; 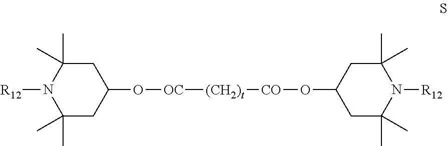
wherein:
t is an integer ranging from 2 to 10;
R₁₂ is as defined above for the compounds of formula R; 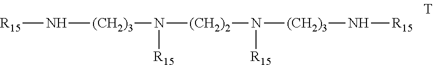
wherein:
R₁₅ is the group of formula (XVI) 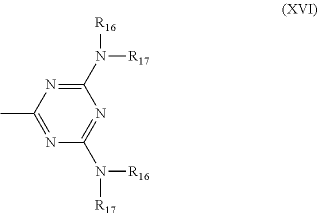
wherein R₁₆ and R₁₇ are independently selected from the group formed by hydrogen, straight or branched C₁-C₄ alkyl groups and the group of formula (XVII) 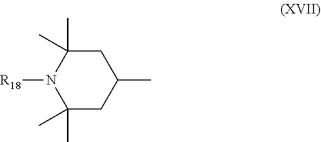
wherein R₁₈ is hydrogen, a straight or branched C₁-C₄ alkyl group or OR₁₉ groups wherein R₁₉ is hydrogen or a straight or branched C₁-C₈ alkyl group; 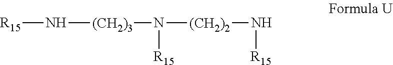
wherein R₁₅ has the meanings defined above for the compounds of formula T.

7. Mixtures according to claim 6 comprising 10% to 90% by weight of the compounds of formula (I).

8. Mixtures according to claim 6 further comprising antioxidants; UV absorbers; nickel stabilisers; plasticisers, lubricants, antistatic agents, flame retardants, corrosion inhibitors, metal deactivators or mineral fillers.

9. Method of stabilizing a polymer with the compounds according to claim 1, said method comprising:
mixing said compounds with said polymer.

10. Method of stabilizing a polymer with the mixture according to claim 6, said method comprising mixing said mixtures with said polymer.

11. The method according to claim 9 wherein said polymer is selected from the group consisting of polyethylene, polypropylene, polystyrene, polybutadiene, polyisoprene and copolymers thereof, polyvinyl chloride, polyvinylidene chloride and copolymers thereof, polyvinyl acetate and copolymers thereof with ethylene; polyesters; polyamides and polyurethanes.

12. The method according to claim 10, wherein said polymer is selected from the group consisting of polyethylene, polypropylene, polystyrene, polybutadiene, polyisoprene and copolymers thereof, polyvinyl chloride, polyvinylidene chloride and copolymers thereof, polyvinyl acetate and copolymers thereof with ethylene; polyesters; polyamides and polyurethanes.
